(19) United States
(12) Patent Application Publication
(10) Pub. No.: US 2004/0143336 A1
(43) Pub. Date: Jul. 22, 2004

(54) Two-piece modular patellar prosthetic system

(76) Inventor: Brian Burkinshaw, Pflugerville, TX (US)

Correspondence Address: ZIMMER TECHNOLOGY, INC., 150 N. WACKER DRIVE, SUITE 1200, CHICAGO, IL, 60606, US

(21) Appl. No.: 10/349,134

(22) Filed: Jan. 22, 2003

(51) Int. Cl. A61F 2/38

(52) U.S. Cl. 623/20.15; 623/20.2

(57) ABSTRACT

A modular patellar prosthetic system used to replace a portion of the natural knee and, more particularly, a two-piece modular patellar prosthetic system having various baseplates and articulation components that are interchangeable with each other. Each baseplate has a fixation surface adapted to engage patellar bone, and each articulation component has a smooth articulation surface. The articulation component and baseplate connect with an attachment mechanism and form an implantable knee prosthesis.

### FIELD OF THE INVENTION

The present invention relates to a modular knee prosthetic system used to replace the natural knee and, more particularly, to a two-piece modular patellar prosthetic system having various baseplates and articulation components that are interchangeable with each other.

### BACKGROUND OF THE INVENTION

In the United States alone, over 200,000 knee replacements are performed each year. Degenerative arthritis, or the gradual degeneration of the knee joint, is the most common reason for these replacements. In this form or arthritis, cartilage and synovium surrounding the knee wear down so underlying bones grind directly on each other.

In knee arthroplasty, portions of the natural knee joint are replaced with prosthetic components. These components include a tibial component, a femoral component, and a patellar component. The femoral component generally includes a pair of spaced condyles that articulate with the tibial component. These condyles form a trochlear groove in which the articulating surface of the patellar component moves. The components are made of materials that exhibit a low coefficient of friction when they articulate against one another.

When the articulating ends of both the femur and tibia are replaced, the procedure is referred to as total knee replacement or TKR. Much effort has been devoted to performing TKR that restores normal, pain-free functions of the knee for the lifetime of the prosthetic components.

Unfortunately, patients can experience problems with the prosthetic knee after a total knee replacement surgery. If a problem occurs, a patient may need a revision surgery wherein some or all of the prosthetic components are replaced. Historically, problems associated with the patellar prosthesis are responsible for as many as 50% of all knee implant revisions. More particularly, complications with the patello-femoral joint or patello-femoral dysfunction are the primary cause of failure in TKR.

One option in a TKR or revision surgery is to implant a prosthetic patellar component. The patellar component has a metallic back or baseplate that is permanently fixed to the patellar bone. Metal baseplates were introduced to provide a more even stress distribution on the natural patella and provide the option for either cement or cementless fixation. An articulation or bearing component is permanently connected to the baseplate to form the prosthetic patellar component. The articulation component is formed from metal or a polymer, such as ultra-high molecular weight polyethylene (UHMWPE).

Despite current advances in the design of prosthetic knees, the patellar component still fails and must be replaced in a revision surgery. Failure of the patellar component occurs for a multitude of reasons. In some instances, the articulation component becomes loose or worn through repeated use. Obviously then, this component must be replaced.

As one disadvantage with current patellar components, replacement of the articulation or bearing component during a revision surgery can be impractical, difficult, or unhealthy for the natural patella. After the initial TKR surgery, the baseplate becomes firmly fixed to the host patellar bone. In present patellar prosthetic designs, the articulation component is permanently attached to the baseplate. So, removal of the articulation component alone is not an option. Instead, both the baseplate and the articulation component must be removed and then replaced. Removing the baseplate from the natural patellar bone is undesirable since healthy bone stock can be damaged or removed from the patella. Further, the stress associated with removing the baseplate during a revision surgery can fracture the natural patella. The patellar bone stock may already be thin or weak, and forcing or prying the baseplate from the bone can damage the patella.

Since removing the baseplate from the patella can have serious, unwanted consequences, surgeons have few options. Manufacturers do not provide modular articulation components that are designed to be removed from the baseplate during a revision surgery. In the past, some attempts have been made to forceably remove or pry apart the articulation component from the baseplate during a revision surgery. Manufacturers, however, would not recommend such a procedure if the components were not designed for this use.

It, therefore, would be advantageous to provide an implantable modular patellar prosthetic system having various baseplates and articulation components that are interchangeable with each other.

### SUMMARY OF THE INVENTION

The present invention is directed toward a modular patellar prosthetic system used to replace a portion of the natural knee and, more particularly, to a two-piece modular patellar prosthetic system having various baseplates and articulation components that are interchangeable with each other.

Each baseplate has a fixation surface and a bearing surface. The fixation surface is adapted to engage patellar bone and includes a plurality of pegs that extend outwardly from the surface to penetrate bone.

Each articulation component has an articulation surface and a bearing surface. The articulation surface has a smooth contour that is adapted to articulate with the femur or femoral prosthesis at the patello-femoral joint. This surface may have various shapes known to those skilled in the art, such as a hyperbolic paraboloid or dome-like configuration. The bearing surface of the articulation component is adapted to engage the bearing surface of the baseplate. In some embodiments, these surfaces are configured to slideably contact or articulate with each other. In other embodiments, the articulation component and baseplate anti-rotationally lock together.

An attachment mechanism couples the baseplate to the articulation component so the bearing surfaces are adjacent each other. The attachment mechanism can have a variety of configurations to enable the articulation component to engage and disengage from the baseplate. In one embodiment, this mechanism includes a peg that protrudes from the bearing surface of the baseplate. The peg has a generally elongated configuration with a circular cross-section. An enlarged head extends at the end of the peg. The articulation component includes a recess shaped to receive the peg. This recess extends into the body of the articulation component and includes a narrow neck region. The neck region deforms to engage with the peg when the two components are connected and deforms to disengage with the peg when the two components are separated.

As one important advantage of the present invention, the articulation component is removeably connectable to the baseplate. In other words, even after the baseplate becomes permanently connected to the patellar bone, an articulation component can be readily attached or detached from the baseplate. During a revision surgery then, healthy bone stock of the natural patella will not be damaged or removed since the baseplate can be left attached to the patella.

As another advantage, an articulation component can be relatively easily removed from or attached to the baseplate. As such, nominal stress is placed on the natural patella as an old articulation component is removed and a new one is attached. The natural patella is thus less likely to fracture or otherwise become damaged during replacement of the articulation component.

As yet another advantage of the invention, multiple articulation components can be easily attached to an implanted baseplate. During a revision surgery then, the implanted articulation component can be removed from the baseplate and replaced with a new, sterile one. Further, multiple articulation components having various sizes and shapes can be attached to the baseplate. As such, the surgeon can choose from a variety of articulation components to meet the specific needs of the patient.

As yet another advantage, a completely assembled modular knee prosthesis of the present invention comprises only two separate or individual components: A base component and an articulation component. No other components are required to form and connect the prosthetic knee. Both the articulation component and the baseplate are formed as a single unit or piece. In other words, these components are not formed from multiple pieces assembled together, but from a unitary, integral unit or piece. Further, these two components include an attachment mechanism that is integrally formed to either or both components. As such, no separate attachment mechanism is required to couple the baseplate and articulation component.

Other objects and advantages of the present invention will be apparent from the following descriptions of a preferred embodiment with reference to the drawings.

### BRIEF DESCRIPTION OF THE DRAWINGS

FIG. 1 is a top perspective view of a modular knee prosthetic system according to the invention and includes a baseplate removeably connectable with three different articulation components.

FIG. 2 is a bottom perspective view of the modular knee prosthetic system of FIG. 1.

FIG. 3 is a side view of the baseplate embedded in patellar bone with the three articulation components of FIG. 1 superimposed on the baseplate to illustrate the different sizes of articulation components.

FIG. 4 is a top perspective view of another modular knee prosthetic system according to the invention and includes a baseplate removeably connectable with three different articulation components.

FIG. 5 is a bottom perspective view of the modular knee prosthetic system of FIG. 4.

FIG. 6 is a top perspective view of yet another modular knee prosthetic system according to the invention showing two different baseplates connectable to six different articulation components.

FIG. 7 is a side view of an alternate embodiment of the baseplate of FIGS. 4 and 5.

FIG. 8 is a top view of the baseplate of FIG. 7.

FIG. 9 is a cross sectional view taken through lines A-A of the baseplate of FIG. 8.

FIG. 10 is a bottom perspective view of an alternate attachment mechanism between a baseplate and articulation component.

FIG. 11 is a top perspective view of FIG. 10.

FIG. 12 is a side perspective view of the baseplate of FIGS. 10 and 11.

FIG. 13 is another side perspective view of the baseplate of FIG. 12 that is rotated 90°.

FIG. 14 is a top view of the base plate of FIGS. 12-13.

FIG. 15 is a top view of the articulation component of FIGS. 10 and 11.

FIG. 16 is a cross sectional view taken along the lines A-A of FIG. 15.

FIG. 17 is a cross sectional view taken along the lines B-B of FIG. 15.

### DETAILED DESCRIPTION OF THE PREFERRED EMBODIMENT

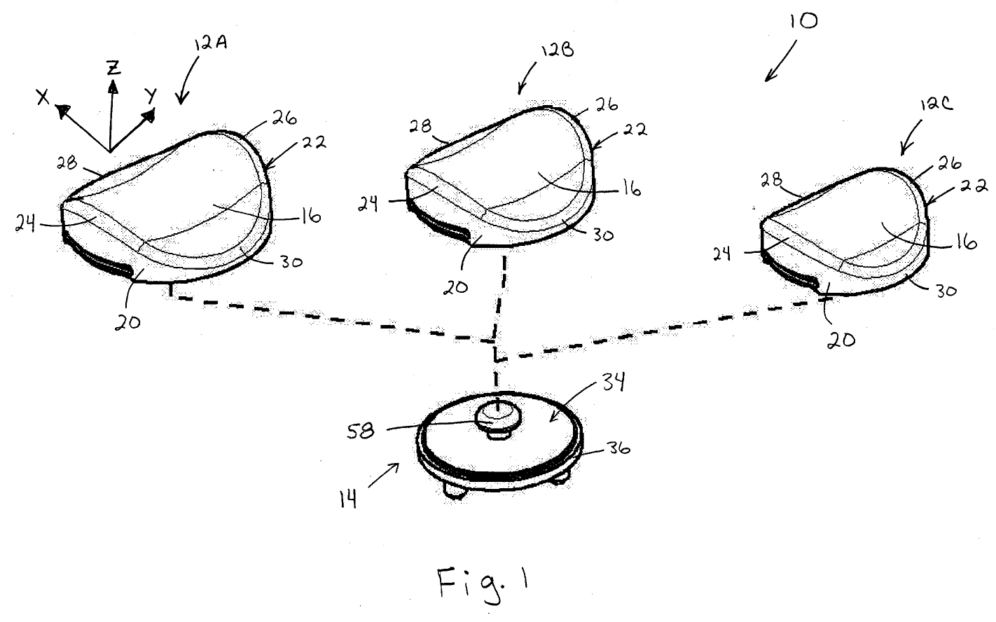

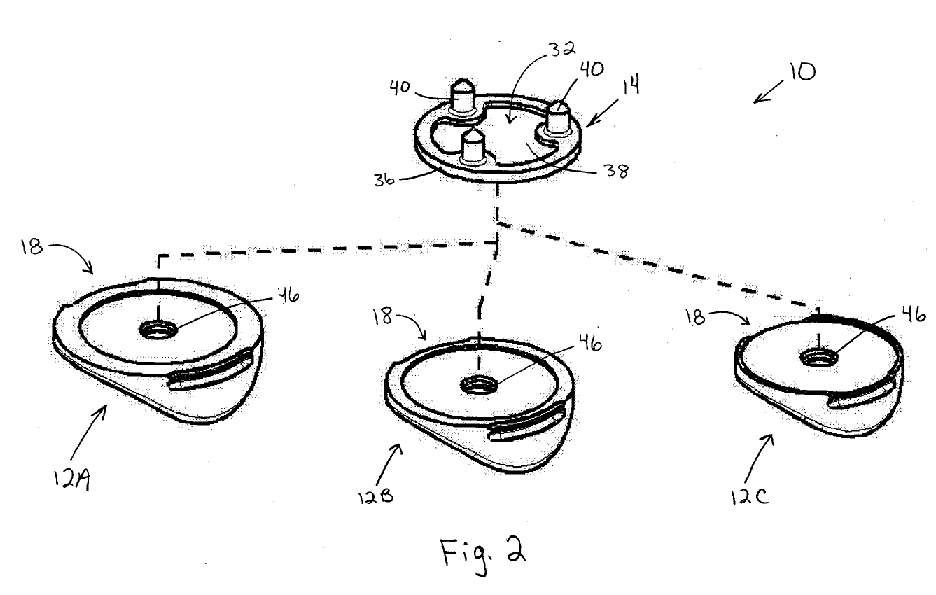

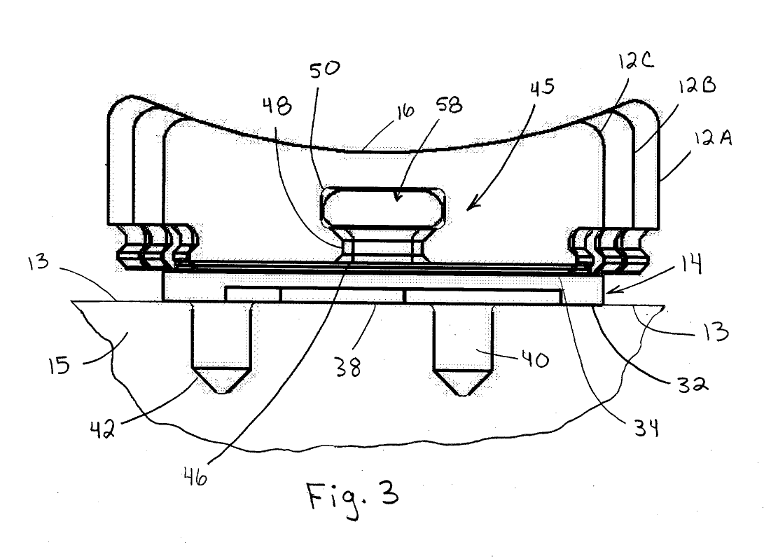

FIGS. 1-3 show a modular knee prosthetic system or kit 10 having a plurality of individual, implantable patellar prostheses. Three different prostheses are shown wherein each prosthesis includes a different articulation or bearing component 12A-12C and a common base component or baseplate 14.

The articulation components and baseplates are shown relative to mutually orthogonal reference axes X, Y and Z (FIG. 1). When a prosthesis is implanted, reference axes X, Y and Z correspond, generally, to well known and accepted anatomical directional terms. The X axis extends generally in the medial-lateral direction, the Y axis extends generally in the inferior-superior direction, and the Z axis extends generally in the posterior-anterior direction. If the prosthesis were implanted on the left patella of a human patient, the ends of each of the X, Y, and Z axes marked with an arrowhead would point generally in the lateral, superior, and posterior directions, respectively.

The present invention may be utilized with various knee surgical techniques and surgeries known to those skilled in the art. As an example, during a TKR surgery, the patella is resected in a plane generally perpendicular to the anterior-posterior direction to remove a posterior portion of the patellar bone, leaving a resected planar bony surface 13 (FIG. 3). When a prosthesis is implanted, the Z axis lies perpendicular to the resected planar bony surface 13 of a patella 15, and the X and Y axes lie parallel to the resected planar bony surface 13.

Articulation component 12 is constructed of a biocompatible material having desirable wear and bearing friction properties, such as biocompatible metals and ultra-high molecular weight polyethylene (UHMWPE). Examples of a suitable materials are Metasul® and Durasul® articulation components manufactured by Centerpulse Orthopedics Inc. of Austin, Tex.

Articulation component 12 includes two primary surfaces: An articulation surface 16 and a planar bearing surface 18 oppositely disposed from the articulation surface. The bearing surface 18 is generally perpendicular to the Z axis and spaced from the articulation surface 16 to define a thickness. A wall 20 extends around the outer perimeter of the articulation component and generally has an elliptical or round shape.

Articulation surface 16, in the preferred embodiment shown, is a hyperbolic paraboloid, also known as a “saddle” shape, in which the intersection of the surface 16 and wall 20 defines an undulating edge 22. Points 24 and 26 are at opposite ends of the “saddle” and designate the locations at which undulating edge 22 is at its maximum spacing from planar bearing surface 18. Points 24 and 26 are on the minor axis of wall 20, and are disposed relative to each other generally in the inferior-superior direction along the Y axis. Points 28 and 30 are at opposite sides of the “saddle” and designate the locations at which undulating edge 22 is at its minimum spacing from planar bearing surface 18. Points 28 and 30 are on the major axis of wall 20, and are disposed relative to each other generally in the medial-lateral direction, along the X axis. Articulation surface 16, so configured, ideally provides congruent sliding contact over an extensive range of articulation between articulation component 12 and the patellar articulation surface of a femoral prosthesis component (not shown) at the patello-femoral joint. Undulating edge 22 at points 24 and 26 at the high ends of the “saddle” functionally defines a ridge that can track the intercondylar groove of the femoral component during flexion and extension of the knee joint.

The saddle shape of the articulating surface provides good contact when mated to the trochlea of the femur. Further, this contact helps to maintain the anatomically “natural” articular bearing motion generated by the normal kinematics of the knee.

Baseplate 14 is constructed of a biocompatible material having desirable wear, bearing friction, and bone engaging properties that are known to those skilled in the art. Examples of such a material are UHMWPE, titanium, titanium alloys, zirconia ceramics, aluminum oxide ceramics, and cobalt chromium alloys.

Baseplate 14 includes a fixation surface 32 for engaging patellar bone 15, a planar bearing surface 34 generally perpendicular to the Z axis and spaced from the fixation surface 32, and an outer wall 36 that extends around the perimeter and is generally parallel to the Z axis. The baseplate generally has an elliptical or round shape to match the size and shape of the articulation component 12.

Fixation surface 32 includes a generally planar surface portion 38 adapted to engage resected planar bony surface 13 generally parallel thereto. The surface portion 38 can be adapted to directly engage and integrate with the patellar bone with or without bone cement. Planar surface portion 38, for example, can include surface texturing to promote osseointegration of baseplate 14. A coating of hydroxyapatite, ceramic, or porous metal are examples of surface texturing known to those skilled in the art. Such coatings can be applied with plasma spraying or sintering techniques. Suitable metals for sintering include titanium and its alloys and cobalt chromium alloys. Other materials and methods for providing a surface that favors osseointegration are well known in the art.

Fixation surface 32 also includes a plurality of pins or pegs 40 that extend downward from the surface. These pegs are evenly and symmetrically spaced apart and are integrally connected to fixation surface 32. The pegs 40 are sized and shaped to be received in correspondingly shaped bores 42 in patella 15 (FIG. 3). Specifically, each peg has a cylindrical body portion with a tapered or conical distal end. One skilled in the art will appreciate that the pegs can have various configurations and textures, such as a straight, ribbed, or tapered shape with macro-textured surface to enhance fixation with bone cement or osseointegration.

One important advantage of the present invention is that the articulation component 12 is removeably connectable to the baseplate 14. Even after the baseplate becomes permanently connected to the patellar bone, an articulation component can be readily or easily attached and detached from the baseplate. The removeable or detachable connection between the baseplate and articulation component provides a modular knee prosthesis. As shown in FIGS. 1-3, three different articulation components 12A-12C can connect to a single baseplate 14. Each articulation component has a similar shape with a different size. FIG. 3 illustrates how each articulation component would fit on the baseplate. FIG. 3 also illustrates the three different sizes of articulation components. Together, the baseplate and plurality of articulation components form a modular knee prosthetic system.

During a TKR or other knee surgery, the surgeon can select any one of various sized and shaped articulation components to connect with a single baseplate. During a revision surgery for example, the implanted articulation component may be damaged, worn, or otherwise need replaced. The articulation component can be easily removed from the baseplate and replaced with a new, sterile one. At the same time though, the baseplate can be left undisturbed and attached to the patellar bone. Thus, a new and different articulation component can be engaged and connected intra-operatively to an existing baseplate previously implanted in the patient.

A coupling or attachment mechanism 45 enables the articulation component 12 and baseplate 14 to be connectable to and removeable from each other. Specifically, in the preferred embodiment, articulation component 12 includes a circular bore or recess 46 that opens from planar bearing surface 18. The recess 46 has a narrow neck portion 48 that leads to an enlarged circular opening or head 50. Further, baseplate 14 includes a pin or peg 58 that is centered on and extending integrally from planar bearing surface 34 in the posterior direction along the Z axis. Pin 58 is circular in cross-section and has a diameter that varies in the profile generally complementarily to the profile of recess 46.

In operation, articulation component 12 and baseplate 14 are configured to engage each other in a removeable lock, snap-retaining relationship. The narrow neck portion 48 of recess 46 deforms elastically under pressure to permit entry of the head of pin 58. After the head of the pin passes into the enlarged opening 50, the neck portion 48 elastically rebounds to engage and to retain pin 58. In order to remove the articulation component from the baseplate, the narrow neck portion 48 of recess 46 deforms elastically under pressure to permit exit of the head of pin 58.

When the pin 58 is engaged in the recess 46, the articulation component 12 can slideably rotate relative to the baseplate 14. More specifically, when articulation component 12 and baseplate 14 are engaged, planar bearing surface 18 of articulation component 12 lies in direct parallel engagement with planar bearing surface 34 of baseplate 14.

FIGS. 1-3 show an attachment mechanism 45 wherein the articulation component 12 has a recess and the baseplate 14 has a peg. One skilled in the art will appreciate that attachment mechanism can be altered without departing from the scope of the invention. As an example, the coupling components of the attachment mechanism can be switched: The articulation component could be configured to have a protruding peg while the baseplate has a recess adapted to receive the peg. Other embodiments as well are within the scope of the invention, and some of these embodiments are shown in the subsequent figures.

Another important advantage of the present invention is that both the articulation component and the baseplate are each formed as a single, unitary piece. In other words, these components are not formed from multiple pieces assembled together, but from a unitary, integral unit or piece. Thus, the articulation component and baseplate are formed from two separate and different pieces that, when connected together, form a prosthetic patellar implant. Further, these two components include an attachment mechanism that is integrally formed to either or both components. As such, no separate attachment mechanism is required to couple the baseplate and articulation component.

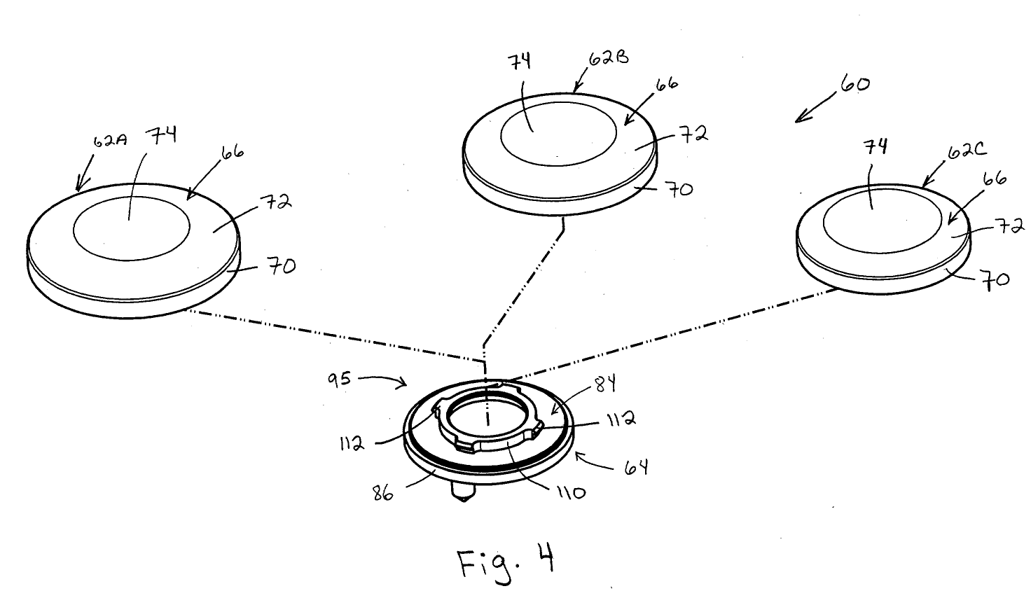

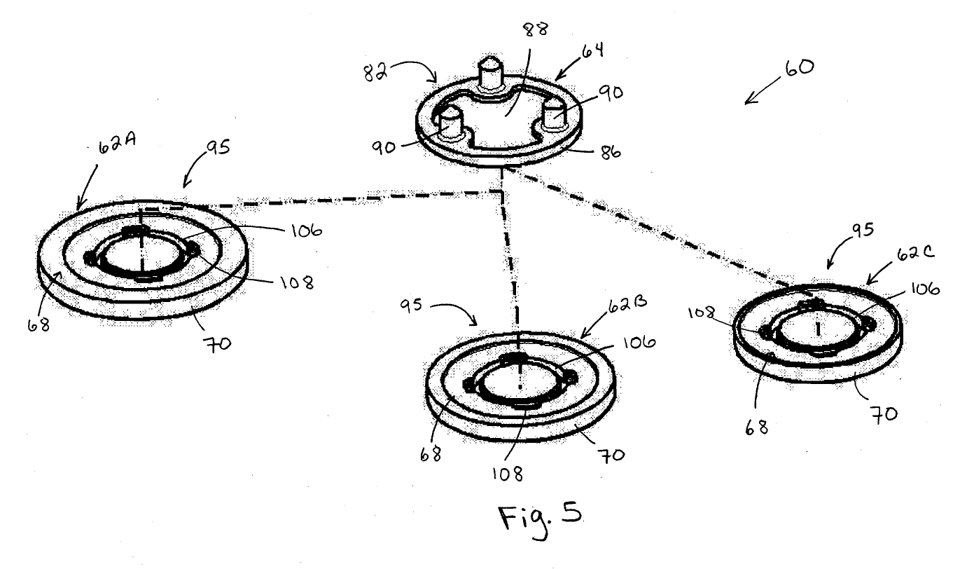

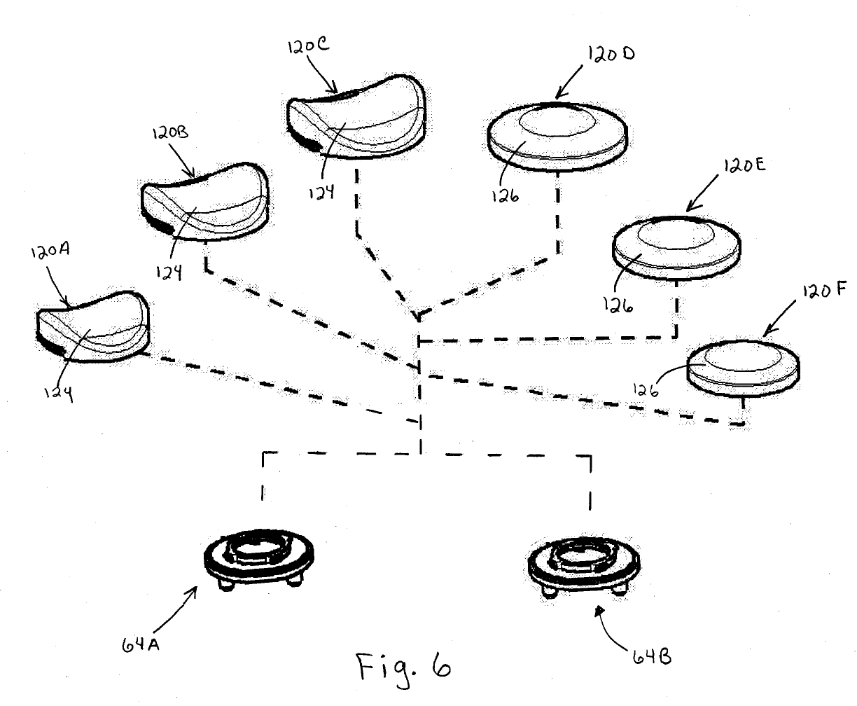

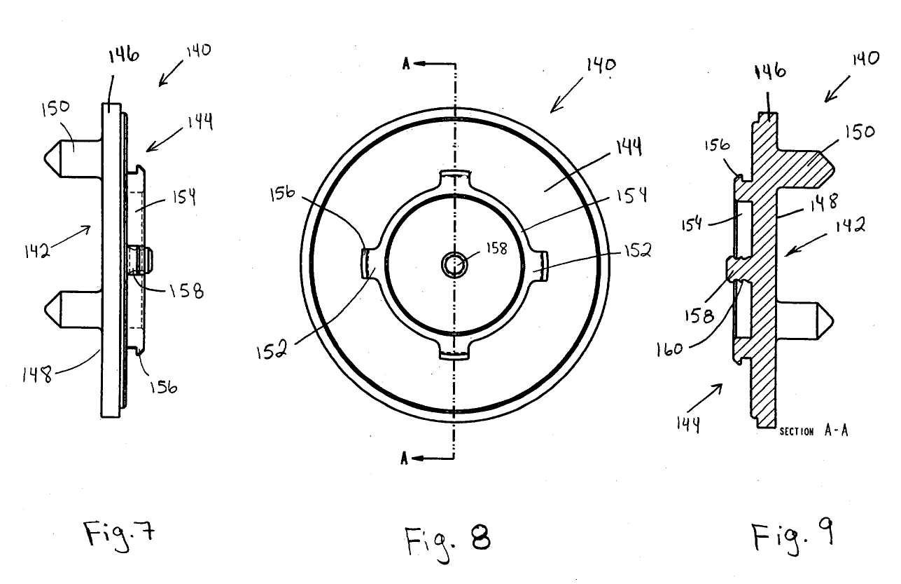

FIGS. 4 and 5 show an alternate modular knee prosthetic system 60 of the present invention. System 60 includes three different articulation components 62A-62C and a common baseplate 64. The system is generally similar to the modular knee prosthetic system 10 discussed in connection with FIGS. 1-3.

Articulation component 62 includes two primary surfaces: An articulation surface 66 and a planar bearing surface 68 oppositely disposed from the articulation surface. The bearing surface 68 is spaced from the articulation surface 66 to define a wall 70 that has a generally round shape that extends around the outer perimeter.

Articulation surface 66 has a smooth outer contour with a generally rounded or dome-shape as shown. The surface has a generally frusto-conical or tapered section 72 that transitions to a generally planar top surface 74. Articulation surface 66 is configured to provide sliding contact over an extensive range of articulation between articulation component 62 and a patellar articulation surface of a femoral prosthesis component (not shown) at the patello-femoral joint.

Baseplate 64 includes a fixation surface 82 for engaging patellar bone and a planar bearing surface 84. The two surfaces are spaced to define a thickness and an outer wall 86 that extends around the perimeter. The baseplate generally has a round shape to match the size and shape of the articulation components 62A-62C.

Fixation surface 82 includes a generally planar surface portion 88 adapted to engage bone and includes a plurality of pins or pegs 90 that extend downward from the surface. These pegs are evenly and symmetrically spaced apart and are integrally connected to fixation surface 82. The pegs 90 are sized and shaped to be received in the patella.

A coupling or attachment mechanism 95 enables the articulation component 62 and baseplate 64 to be connectable to and removeable from each other. Specifically, articulation component 62 includes a circular channel 106. The channel has a rectangular cross-section and includes four rectangular recesses 108. The baseplate 64 includes a circular protrusion 110 that extends outwardly from the bearing surface 84. The protrusion 110 has a rectangular cross-section with four rectangular legs 112. The protrusion 110 is shaped and adapted to be received in the channel 106 of the articulation component 62.

In operation, articulation component 62 and baseplate 64 are configured to engage each other in a locking relationship such that the two components can be connected and removed from each other. The protrusion 110 extends into the channel 106 so legs 112 engage and lock into recesses 108. When articulation component 62 and baseplate 64 are engaged, planar bearing surface 68 of articulation component 62 lies in direct parallel engagement with planar bearing surface 74 of baseplate 64.

As shown in FIGS. 4 and 5, any one of three different articulation components 62A-62C are engageable with and removable from a single baseplate 64. Each articulation component has a similar shape but has a different size. Three different sizes are shown, such as large, medium, and small sizes. One skilled in the art will appreciate that the number of sizes and shapes can increase to offer a more diversified modular prosthetic knee system.

FIG. 6 shows that a plurality of baseplates 64A and 64B with different sizes can be connected to various articulation components 120A-120F. Six different articulation components are shown. Components 120A-120C have a saddle shape articulation surface 124 similar to the surface shown and described in connection with FIGS. 1-3. By contrast, components 120D-120F have a rounded shape articulation surface 126 similar to the surface shown and described in connection with FIGS. 4 and 5.

FIG. 6 illustrates the adaptability of the present invention. A plurality of differently sized and shaped baseplates can be connected to a variety of differently sized and shaped articulation components. Each of the articulation components can be connected and removed from each of the baseplates to form a modular prosthetic knee system.

FIGS. 7-9 show an alternate baseplate 140 that has a configuration generally similar to the baseplate 64 described in FIGS. 4 and 5. Baseplate 140 includes a fixation surface 142 for engaging patellar bone and a planar bearing surface 144. The two surfaces are spaced to define a thickness and an outer wall 146 that extends around the perimeter. The baseplate generally has a round shape to match the size and shape of the articulation components described in FIGS. 4 and 5. Fixation surface 142 includes a generally planar surface portion 148 adapted to engage bone and includes a plurality of pins or pegs 150 that extend downward from the surface. These pegs are evenly and symmetrically spaced apart and are integrally connected to fixation surface 142. The bearing surface 144 includes a circular protrusion 154 that extends outwardly from the bearing surface 144. The protrusion 154 has a rectangular cross-section with four rectangular legs 152. Each leg has a lip 156 at a distal tip. Further, a cylindrical peg 158 extends outwardly from the bearing surface. The peg has a plurality of outer ribs 160. The protrusion 154 and peg 158 are shaped and adapted to be received in and lockably engage with a corresponding channel and recess of an articulation component.

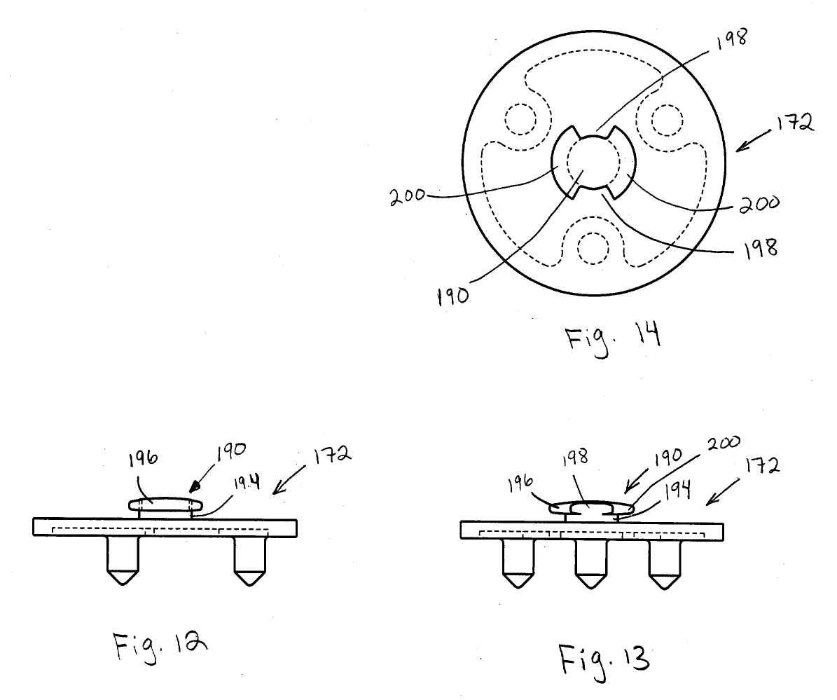

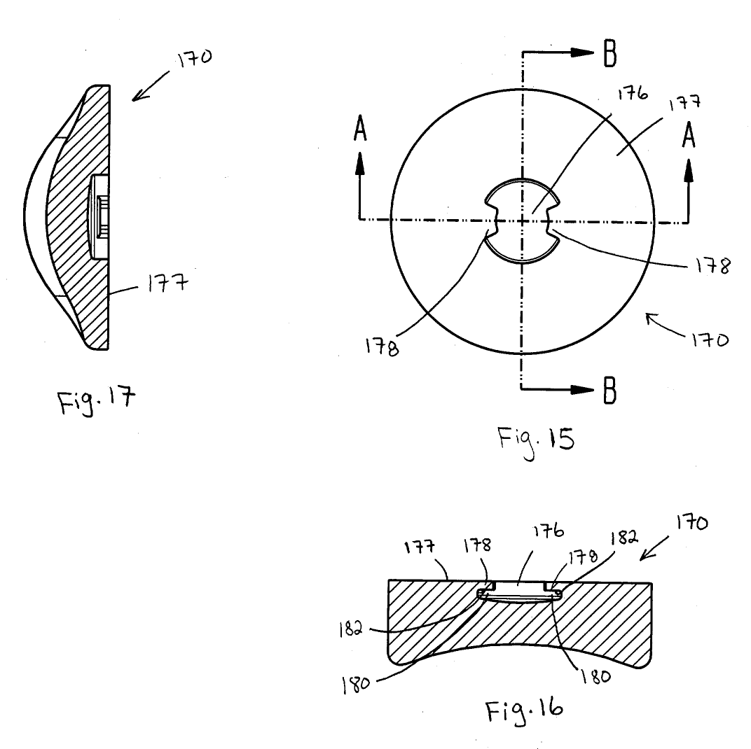

FIGS. 10-17 illustrate an alternate embodiment for an articulation component 170 and a baseplate 172. These components are generally similar to the articulation components and baseplate shown and described in connection with FIGS. 1-3, and the similarities will not be described. One important difference resides in the configuration of the attachment mechanism 174.

As best shown in FIGS. 15-17, articulation component 172 includes a circular bore or recess 176 along the bearing surface 177. Two lips or shoulders 178 are oppositely disposed and extend into the recess at the opening. The shoulders 178 do not extend completely into the recess and form a channel 180 under the bottom surface 182 of each shoulder.

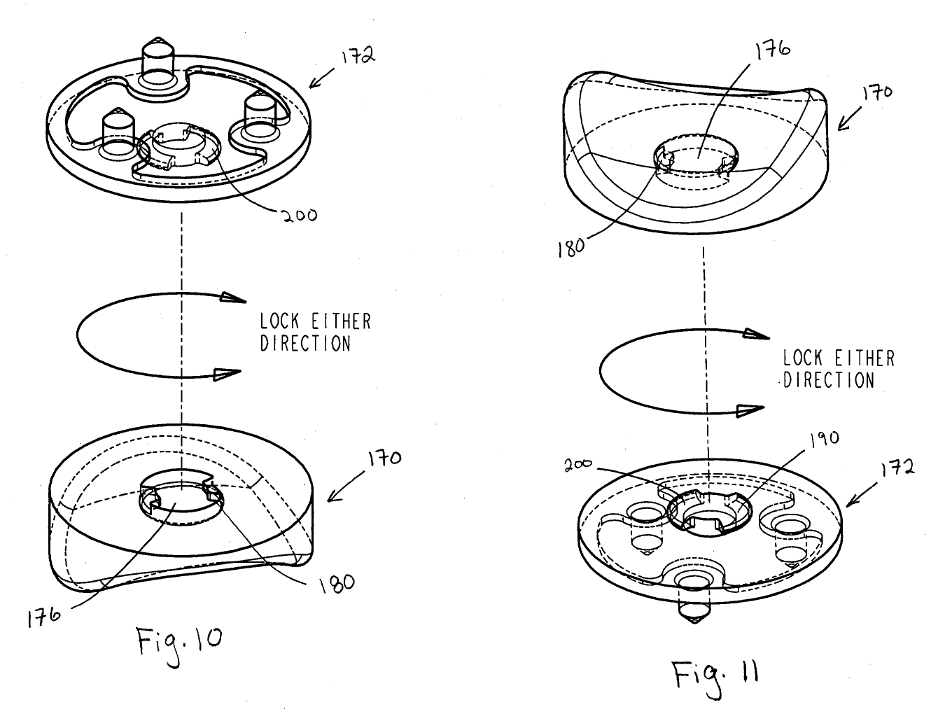

As best shown in FIGS. 12-14, baseplate 172 includes a protrusion or peg 190 extending from the bearing surface 192. The peg 190 has a cylindrical portion 194 and a head portion 196. This head has two cutouts 198 that are oppositely disposed from one another and two arms or wings 200 that-are oppositely disposed from one another.

In operation, articulation component 172 and baseplate 174 are configured to engage each other in a locking relationship such that the two components can be connected and removed from each other. The peg 190 extends into the recess 176 when the shoulders 178 are aligned with the cutouts 198. As illustrated in FIGS. 10 and 11, once the peg 190 is inserted into recess 176, the articulation component can be rotated 90° in either a clockwise or counterclockwise direction. After the rotation, the wings 200 of peg 190 are positioned into the channels 180. In this position, the articulation component is locked to the baseplate. In order to remove the articulation component from the baseplate, the articulation component can be rotated 90° in either a clockwise or counterclockwise direction. After the rotation, the wings 200 of peg 190 are disengaged from channels 180. In this position, the articulation component is unlocked and can be lifted from the baseplate.

As described in FIGS. 1-17, the articulation component enjoys a single degree of freedom of movement relative to the baseplate. The term “degree of freedom” is used in its ordinary engineering sense to mean freedom of a component to rotate about or translate along a line that is parallel to one axis of a three-axis Cartesian coordinate system fixed in orientation relative to the reference component. The freedom to rotate about such a line comprises one degree of rotational freedom, and the freedom to translate along such a line comprises one translational degree of freedom. A component can enjoy a maximum of six degrees of freedom, in which case the component can rotate about any axis and can translate along any axis. Essentially, a component with six degrees of freedom is unconstrained by any other component.

The present invention is equally utilized with one or several degrees of freedom. U.S. Pat. No. 5,702,465 entitled “Patella Prosthesis Having Rotational and Translational Freedom” is incorporated herein by reference and teaches an articulation component and baseplate having two degrees of freedom. The present invention can be employed with the embodiments taught therein.

Further, the present invention can be utilized with various prosthetic knee designs, including both mobile bearing and fixed knee designs.

Even further, one skilled in the art will appreciate that the attachment mechanism used to connect the articulation component to the baseplate may be modified without departing from the scope of the invention. For example, the male and female components on the articulation component could be switched with the corresponding components on the baseplate.

Further yet, the FIGS. 1-5 and 7-17 illustrate a single baseplate that is connectable to a plurality of differently sized and shaped articulation components. Multiple baseplates with different sizes and shapes (including different thicknesses), though, are contemplated for use with the present invention. The invention includes a family of baseplate components and a family or articulation components that can be produced and packaged separately or together with the intention of producing a modular prosthetic knee system. The articulation components and baseplates can be assembled intra-operatively in a mix and match fashion to meet the needs of the patient. Further, the present invention contemplates multiple components in a family of articulation components and baseplates that can be removed or replaced with like or different components from the family. A large family of components can serve a wide array of patient needs and give the surgeon modularity between components even during intra-operative assembly.

Although illustrative embodiments have been shown and described, a wide range of modifications, changes, and substitutions is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1) A modular patellar prosthetic system adapted to replace a portion of a natural patella, the system comprising:
a baseplate having a fixation surface adapted to engage the natural patella and a bearing surface oppositely disposed from the fixation surface; and
a plurality of articulation components, each articulation component has an articulation surface adapted to articulate with a femoral component at a patello-femoral joint and a bearing surface adapted to slideably articulate with the bearing surface of the baseplate, wherein each articulation component has a different size and is adapted to attach and detach from the baseplate and is formed as a single, unitary unit.

2) The modular patellar prosthetic system of claim 1 wherein the articulation surfaces of the articulation components include at least two different shapes.

3) The modular patellar prosthetic system of claim 2 wherein at least one articulation surface has a saddle shape and at least another articulation surface has dome-shape with a generally planar surface.

4) The modular patellar prosthetic system of claim 1 wherein six different articulation components are provided, and three of the articulation components have an articulation surface with a saddle shape and three of the articulation components have an articulation surface other than a saddle shape.

5) The modular patellar prosthetic system of claim 1 wherein the baseplate is formed from a single unitary unit.

6) The modular patellar prosthetic system of claim 5 wherein the baseplate and one of the articulation components connect together to form a prosthetic patellar implant formed from two separate and different pieces.

7) The modular patellar prosthetic system of claim 6 wherein the bearing surface of the articulation component slideably rotates on the bearing surface of the baseplate while the articulation component is connected to the baseplate.

8) A modular patellar prosthetic system, comprising:
at least one baseplate integrally formed as a single unit and having a fixation surface adapted to engage natural patellar bone, a bearing surface oppositely disposed from the fixation surface, and an attachment mechanism located on the bearing surface; and
a plurality of articulation components, each articulation component integrally formed as a single unit and having a smooth articulation surface adapted to articulate with a femoral component at a patello-femoral joint and having a bearing surface with an attachment mechanism that is adapted to engage directly the attachment mechanism of the baseplate, wherein each articulation component can be attached and reattached to the baseplate.

9) The modular patellar prosthetic system of claim 8 wherein the plurality of articulation components includes at least two articulation components with different shapes and different sizes.

10) The modular patellar prosthetic system of claim 9 wherein one articulation component has a saddle shape and one articulation component has a shape other than a saddle shape.

11) The modular patellar prosthetic system of claim 10 wherein one articulation component has a dome-shape.

12) The modular patellar prosthetic system of claim 8 wherein the attachment mechanism on the baseplate and articulation components removeably couple together to provide modularity between the baseplate and plurality of articulation components.

13) The modular patellar prosthetic system of claim 8 wherein the articulation components are adapted to be connected to and removed from the baseplate while the baseplate is permanently affixed to natural patellar bone.

14) The modular patellar prosthetic system of claim 13 wherein the attachment mechanism on the articulation components deforms as the baseplate is connected to and removed from the articulation components.

15) The modular patellar prosthetic system of claim 14 wherein the attachment mechanisms on the articulation components and baseplate snap-fit together.

16) A modular patellar prosthetic system adapted to replace a portion of a natural patella, the system comprising:
a plurality of baseplates, each baseplate has a fixation surface adapted to affix to the natural patella and a bearing surface oppositely disposed from the fixation surface, wherein at least two baseplates are provided with different sizes; and
a plurality of articulation components, each articulation component has an articulation surface adapted to articulate with a femoral component at a patello-femoral joint and a bearing surface adapted to engage the bearing surface of the baseplates, wherein at least two articulation components are provided with different sizes and each articulation component is adapted to attach and detach from each of the baseplates.

17) The modular patellar prosthetic system of claim 16 wherein the articulation components are attachable and detachable from a baseplate while the baseplate is permanently affixed to the patella.

18) The modular patellar prosthetic system of claim 17 wherein the articulation components removeably snap-fit to the baseplates.

19) The modular patellar prosthetic system of claim 16 wherein the plurality of articulation components includes two articulation components with a saddle shape and two articulation components with a dome-shape.

20) The modular patellar prosthetic system of claim 19 wherein each articulation component has a different size.
